(19) United States
(12) Patent Application Publication
(10) Pub. No.: US 2010/0114275 A1
(43) Pub. Date: May 6, 2010

(54) IMPLANTABLE MEDICAL LEAD INCLUDING WINDING FOR IMPROVED MRI SAFETY

(75) Inventor: Xiaoyi Min, Thousand Oaks, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/261,977

(22) Filed: Oct. 30, 2008

(51) Int. Cl. A61N 1/05

(52) U.S. Cl. 607/116; 29/825

(57) ABSTRACT

An implantable medical lead for coupling to an implantable pulse generator may be configured for improved MRI safety. The lead may include: a tubular body including a proximal end and a distal end; a first electrode operably coupled to the tubular body near the distal end; and a first electrical coil conductor extending distally through the body from the proximal end and electrically connected to the first electrode. The coil conductor may include at least one transition in which the coil conductor changes from being helically coiled in a first direction to being helically coiled in a second opposite direction. A method of forming such a lead may include: helically coiling at least a portion of a first electrical coil conductor by winding the coil conductor in a first direction, and winding the coil conductor in a second direction opposite the first direction so as to form a transition.

### CROSS-REFERENCE TO RELATED APPLICATION

The present application contains subject matter that is related to the following copending U.S. patent applications: 1) Ser. No. 12/110,150, filed Apr. 25, 2008, titled “Implantable Medical Lead Configured for Improved MRI Safety” (Attorney Docket A08P1014); 2) Ser. No. 11/932,030, filed Oct. 31, 2007, titled “Implantable Medical Lead Configured for Improved MRI Safety” (Attorney Docket A07P1164); and 3) Ser. No. 12/197,957, filed Aug. 25, 2008, titled “MRI Compatible Lead” (Attorney Docket A08P1034). The entire disclosures of these related applications are incorporated herein by reference.

### FIELD OF THE INVENTION

The present invention relates to medical methods and apparatus. More specifically, the present invention relates to implantable medical leads and methods of manufacturing and utilizing such leads.

### BACKGROUND OF THE INVENTION

Existing implantable medical leads for use with implantable pulse generators, such as neurrostimulators, pacemakers, defibrillators or implantable cardioverter defibrillators (“ICD”), are prone to heating and induced current when placed in the strong magnetic (static, gradient and RF) fields of a magnetic resonance imaging (“MRI”) machine. The heating and induced current are the result of the lead acting like an antenna in the magnetic fields generated during an MRI. Heating and induced current in the lead may result in deterioration of stimulation thresholds or, in the context of a cardiac lead, even increase the risk of cardiac tissue damage and perforation.

Over fifty percent of patients with an implantable pulse generator and implanted lead require, or can benefit from, an MRI in the diagnosis or treatment of a medical condition. MRI modality allows for flow visualization, characterization of vulnerable plaque, non-invasive angiography, assessment of ischemia and tissue perfusion, and a host of other applications. The diagnosis and treatment options enhanced by MRI are only going to grow over time. For example, MRI has been proposed as a visualization mechanism for lead implantation procedures.

There is a need in the art for an implantable medical lead configured for improved MRI safety. There is also a need in the art for methods of manufacturing and using such a lead.

### SUMMARY

Disclosed herein is an implantable medical lead for coupling to an implantable pulse generator and configured for improved MRI safety. In particular, embodiments disclosed herein may improve MRI safety by reducing or even canceling induced currents in medical leads. Such reduction and/or canceling may reduce or even eliminate risks of stimulation and/or heating resulting from exposure of medical leads to magnetic and/or electrical fields.

In one embodiment, the lead may include a tubular body, a first electrode and a first electrical coil conductor. The first electrode may be operably coupled to the tubular body near the distal end. The first electrical coil conductor may extend distally through the body from the proximal end and may electrically connect to the first electrode. The first coil conductor may also include at least one transition in which the first coil conductor changes from being helically coiled in a first direction to being coiled in a second opposite direction.

Disclosed herein is a method of forming an implantable medical lead configured for improved MRI safety. In particular, embodiments disclosed herein may produce a lead that improves MRI safety by reducing or even canceling induced currents in medical leads.

In one embodiment, the method may include: helically coiling at least a portion of a first electrical coil conductor by winding the first coil conductor in a first direction, and winding the first coil conductor in a second direction opposite the first direction so as to form a transition in the first coil conductor in which the first coil conductor changes from being helically coiled in the first direction to being helically coiled in the second direction. The method may also include forming an implantable medical lead including the helically coiled first coil conductor.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of a lead and a pulse generator for connection thereto.

FIG. 2 is an isometric view of a longitudinal segment of the lead tubular body in the vicinity of arrow A in FIG. 1, wherein the coil conductor is formed to include a transition according to one embodiment.

FIGS. 3A-C illustrate induced currents in a portion of the coil conductor near the transition in the helical coiling of the coil conductor that may result from exposing the coil conductor to electrical and/or magnetic fields.

FIG. 4 is an isometric view of a longitudinal segment wherein the coil conductor is formed to include two transitions according to another embodiment.

FIG. 5 is an isometric view of a longitudinal segment wherein the coil conductor is formed to include a plurality of transitions according to another embodiment.

FIG. 6 an isometric view of a longitudinal segment wherein two coil conductors are formed to include a plurality of transitions according to one embodiment.

### DETAILED DESCRIPTION

Disclosed herein is an implantable medical lead 10 configured for improved MRI safety. In various embodiments, the lead 10 may include conductors and/or conductor arrangements configured to reduce, if not totally eliminate, the potential for MRI induced currents and heating in conductors extending through the lead body to electrodes, such as those used for pacing, sensing and/or defibrillation.

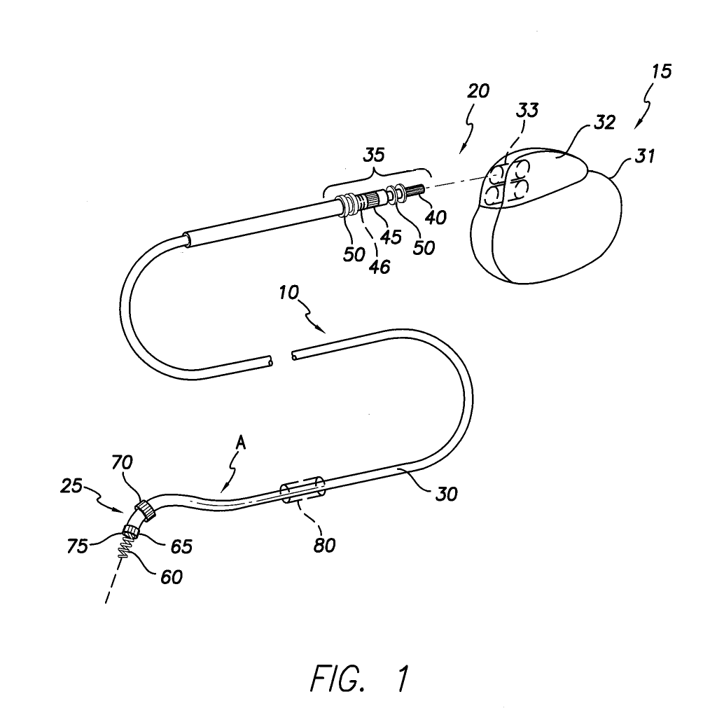

For an overview discussion regarding an embodiment of a lead 10 configured for improved MRI safety, reference is made to FIG. 1, which is an isometric view of such a lead 10 and a pulse generator 15 for connection thereto. As shown in FIG. 1, the pulse generator 15, which may be a neurrostimulator, pacemaker, defibrillator or ICD, may include a housing 31 and a header 32. The housing 31 may enclose the electrical components of the pulse generator 15. The header 32 may be mounted on the housing 31 and may include lead receiving receptacles 33 for connecting one or more leads 10 to the pulse generator 15.

As illustrated in FIG. 1, in one embodiment, the lead 10 may include a proximal end 20, a distal end 25 and a tubular body 30 extending between the proximal and distal ends. The proximal end 20 may include a lead connective end 35 having a pin contact 40, a first ring contact 45, a second ring contact 46, which is optional, and sets of spaced-apart radially projecting seals 50. In other embodiments, the lead connective end 35 may include a greater or lesser number of contacts and may include the same or different types of seals. The lead connective end 35 may be received in one of the lead receiving receptacles 33 of the pulse generator 15 such that the contacts 40, 45, 46 electrically contact corresponding electrical terminals within the respective receptacle 33 and the seals 50 prevent the ingress of body fluids into the respective receptacle 33.

As depicted in FIG. 1, in one embodiment, the lead distal end 25 may include a distal tip 55, an anchor 60, a tip electrode 65, and a ring electrode 70. The anchor 60 may be extendable from an orifice in the distal tip 75. The tip electrode 65 may form the distal tip 75 of the lead body 30, and the ring electrode 70 may extend about the circumference of the lead body 30 proximal of the tip electrode 65. In other embodiments, there may be a greater or lesser number of electrodes 65, 70 in similar or different configurations. Also, the anchor 60 may or may not have other configurations and may or may not also serve as an electrode.

As indicated in FIG. 1, the lead 10 may include an optional defibrillation coil 80, which may extend about the circumference of the lead body 30. The defibrillation coil 80 may be located proximal of the ring electrode 70.

In one embodiment, the tip electrode 65 may be in electrical communication with the pin contact 40 via electrical conductors, the ring electrode 70 may be in electrical communication with the ring contact 45 via other electrical conductors, and the defibrillation coil 80 may be in electrical communication with the second ring contact 46 via yet other conductors. The various conductors may extend through the lead body 30 and are described later in this Detailed Description.

But for the novel conductor configurations discussed below, the conductors could act as an antenna in the magnetic field of an MRI. As a result, current could be induced in the conductors, causing the conductors and the electrodes connected thereto to stimulate and/or heat and potentially damage the lead and/or tissue contacting the electrodes.

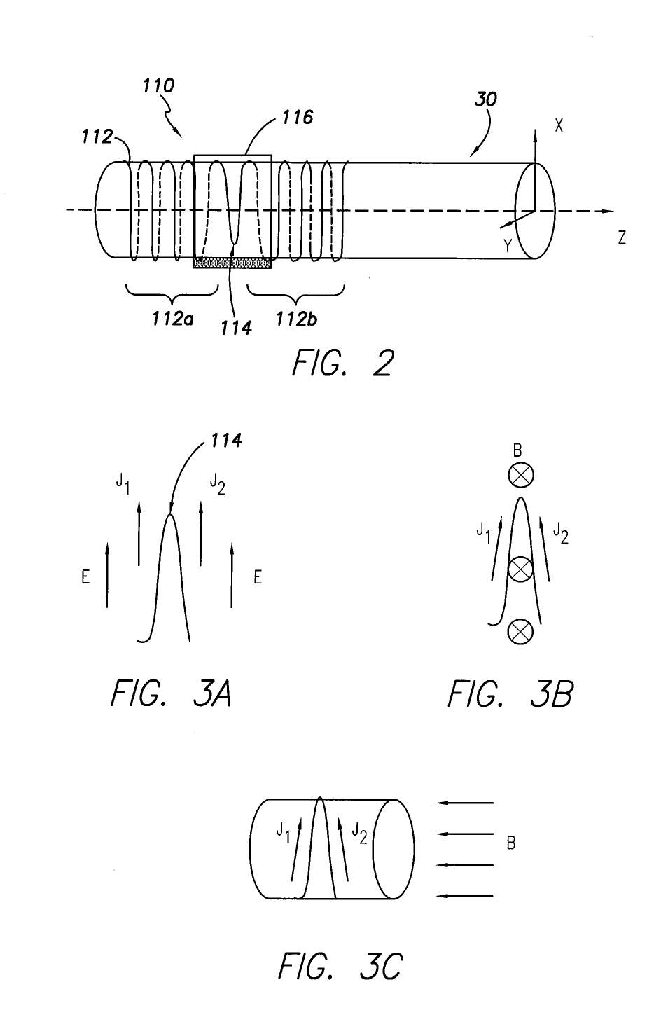

For a discussion of an embodiment of the lead 10 configured to reduce, if not totally eliminate, current induction and heating caused in lead conductors subjected to MRI, reference is made to FIGS. 2 and 3A-C. FIG. 2 is an isometric view of a longitudinal segment of the lead tubular body 30 in the vicinity of arrow A in FIG. 1, wherein the body 30 is represented schematically only for the sake of reference, without regard to the layers of the body 30, to illustrate a coil conductor arrangement 110. FIGS. 3A-C illustrate induced currents that may result from exposure of the lead 10 to a MRI.

As shown in FIG. 2, the coil conductor arrangement 110 may comprise an electrical coil conductor 112. The coil conductor 112 may be formed of an electrically conductive material, such as MP35N, silver-cored MP35N, tantalum, etc. The coil conductor 112 may be formed of a single filar or multiple filars, for example, having a diameter of between approximately 0.002″ and approximately 0.01″. Also, the coil conductor 112 may be insulated or not. For example, the lead body 30 may include one or more insulation layers, as appropriate or desired. The insulation may be of any suitable material, such as silicone rubber, polyurethane, silicone rubber-polyurethane-copolymer (“SPC”), etc. In general, the coil conductor 112 may be any known or hereafter developed conductor that is suitable for use in an implantable medical lead.

As shown in FIG. 2, the coil conductor 112 may be helically wound or coiled for a portion of its length. For example, the coil conductor 112 may be helically wound for one or more discrete portions of its length or over its entire length, as appropriate or desired. Only a portion of the coil conductor is shown in FIG. 2 to illustrate particular inventive features. However, it should be understood that the coil conductor extends the length of the body 30, for example, to provide electrical communication between one or more of the contacts 40, 45, 46 and one or more of the electrodes 65, 70, 80.

The coil conductor 112 may include one or more transitions 114 at which the direction of the helical coiling changes direction. For example, a first portion 112a of the coil conductor 112 may be coiled counterclockwise as viewed axially from the right side of FIG. 2. A second portion 112b of the coil conductor 112 after the transition 114 may be coiled in an opposite direction, that is, clockwise as viewed axially from the right side of FIG. 2. It should be understood that the direction of coiling for the first and second portions 112a, 112b may be reversed from that shown. In general, each transition 114 in the coil conductor 112 is a region in which the direction of the coiling changes. Thus, as illustrated for other embodiments discussed herein, the coil conductor 112 may include multiple transitions 114 in which the direction of coiling changes from clockwise to counter clockwise and vice versa. The coil conductor configurations with the transitions 114 disclosed herein may be applied to various lead configurations such as coaxial leads, co-radial leads, and etc.

Referring to FIGS. 3A-C, the transition 114 may reduce, if not totally eliminate, current induction and heating caused in the coil conductor 112 by exposure to electrical and/or magnetic fields, such as with a MRI. For example, as indicated in FIG. 3A, an electrical field E along the conductors or, more particularly, the component of the electrical field E extending along the axis of the lead may cause induced currents J₁ and J₂ to flow in the coil conductor in opposite directions on each side of the transition 114, with the direction of the helical coiling determining the direction of the respective induced current. If the lengths of the first and second portions 112a, 112b (shown in FIG. 2) are equal or substantially equal, then the induced currents may be equal or substantially equal and may cancel each other entirely.

Similarly, a magnetic field B in a direction perpendicular to the axis of the lead, as shown in FIG. 3B, or a magnetic field B in an axial direction, as shown in FIG. 3C, may cause induced currents J₁ and J₂ to flow in the coil conductor in opposite directions on each side of the transition 114, again with the direction of the helical coiling determining the direction of the respective induced current. Thus, the induced currents J₁ and J₂ may substantially, if not entirely, cancel each other.

Referring back to FIG. 2, a reinforcement ring 116 may be disposed over the transition 114. The reinforcement ring 116 may be made of a metal material or a non-metal material such as PEEK, etc. The reinforcement ring 116 may be imbedded in or form a portion of the lead body jacket layers extending over the conductors and forming the outer surface of the lead body. The reinforcement ring 116 may be positioned to extend over the transition 114 and for a distance to either side of the transition to reinforce the lead body in the vicinity of the transition. This reinforcement may or may not be desirable depending on the extent to which the transition changes the mechanical properties of the lead body as compared to the regions of the lead body not having a transition. It should be understood that the transition 114 may be defined as a region in which the change from coiling in one direction to another occurs. As conductor may have a single transition 114 or multiple transitions 114 as it extends longitudinally along the lead. While a transition 114 may be substantially formed of two oppositely wound coils or loops intersecting each other to provide a reverse in the winding direction, some transitions 114 may further include a substantially axially extending portion that is not coiled in either direction, thereby forming the junction or intersection between the two oppositely wound coils. The use of a reinforcement ring 116 may provide a mechanical structure for the transition point protecting breakage of the lead or conductor of the coil during motion or deflection of the lead. The width of the ring 116 may be 1-5 mm and thickness may be in a range of 4-10 mils.

As can be understood from FIG. 2, in one embodiment, the first portion 112a of the coil conductor 112 is helically coiled at a first angle a relative to a normal projection from the Z axis. The second portion 112b of the coil conductor is helically coiled at a second angle β relative to a normal projection from the Z axis. In relation to the X axis projection in FIG. 2, the first angle α is a negative angle and the second angle β is a positive angle. Thus, as can be understood from FIG. 2, the helical angle of the coil conductor 112 changes in the transition 114 from negative to positive or vice versa. Depending on the embodiment, the magnitude of the helical angle may remain constant or may change moving across the transition 114.

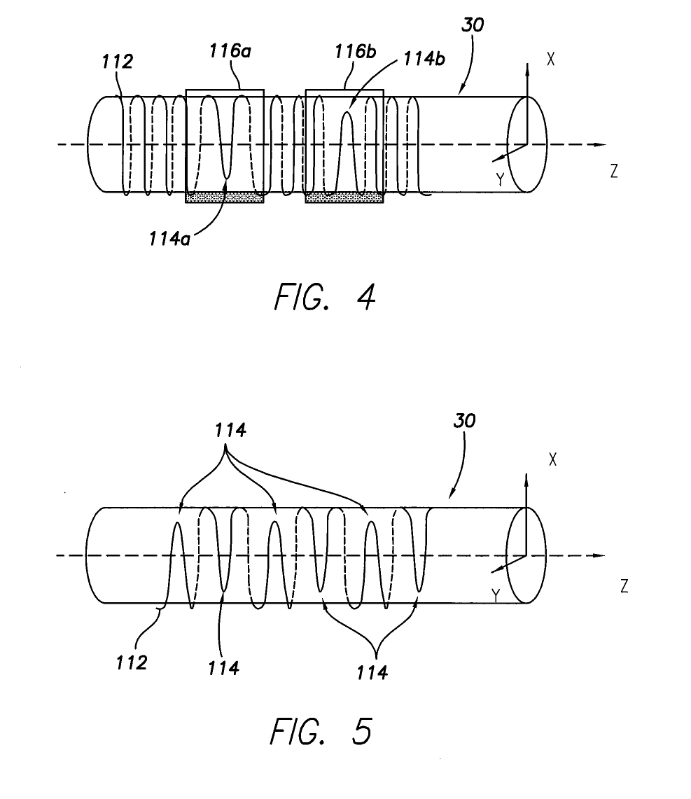

As discussed above, the coil conductor may include more than one transition 114. FIG. 4 illustrates an embodiment in which two transitions 114a and 114b are provided. In this example, the transitions 114 are spaced apart by less than five windings or complete turns. The spacing between adjacent transitions 114 may determine the length of the portions of the coil conductor before and after each transition 114 and thus the magnitude of the current induced in the portions of the conductor 112 before and after each transition. Conversely, the length of the coil conductor 112 (or the length of coiled portions of the coil conductor 112) may determine the suitable number of transitions 114 and/or spacing between adjacent transitions 114. The spacing between adjacent transitions 114 may be wavelength dependent. The wavelength along the wire may depend on parameters such as, for example, coil pitch, coil diameter, wire insulation thickness, etc.

Depending on the embodiment, a coil may have one, two, three or more transitions 114 between the distal and proximal ends of the coil. The spacing of the transitions along the length of the coil may be generally uniform or equal, or the non-uniform or unequal. Depending on the embodiment, reinforcement rings 116a, 116b may be provided for each respective transition 114a, 114b, some of the transitions or none of the transitions.

FIG. 5 illustrates another embodiment in which the coil conductor 112 includes a plurality of transitions 114. As shown, the transitions 114 may be periodic, for example, with the helical pitch length and/or length of portions of the coil conductor 112 before and after each transition 114 being equal. Also, the transitions 114 may be spaced apart by less than 1.5 windings. This may help to minimize the magnitude of the induced currents prior to canceling at the transitions 114, thereby minimizing any resulting heating. While the embodiment depicted in FIG. 5 is shown without reinforcement rings 116, in other such embodiments, the reinforcement rings 116 may be provided for some or all transitions 114. Where reinforcement rings 116 are not provided, the shrink tubing or other reinforcing layers may be extended over the regions including the transitions 114 to reinforce the lead body.

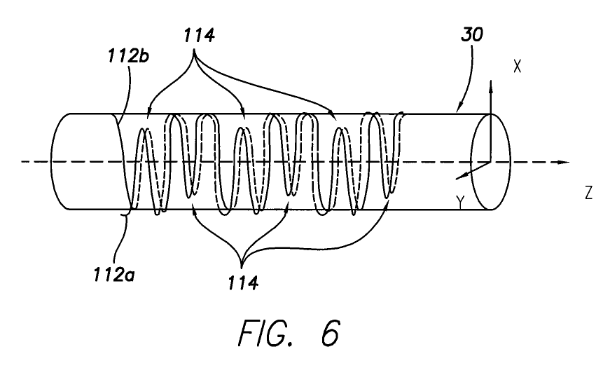

It should be understood that the transitions may be employed in a plurality of separate coil conductors and/or a plurality of filars. For example, FIG. 6 illustrates an embodiment in which a first coil conductor 112a includes a plurality of transitions 114 and a second coil conductor 112b includes a plurality of transitions. The first and second coil conductors 112a, 112b may be coaxial or co-radial, oppositely wound wires as shown, as appropriate or desired. Further, the first and second coil conductors 112a, 112b may be connected to the same or different electrodes and/or contacts.

As shown in FIG. 6, the transitions 114 of the second coil conductor 112b may be axially aligned or substantially axially aligned with the transitions 114 of the first coil conductor 112a. This may allow a same reinforcement ring (not shown in FIG. 6) to be used for a transition 114 of the first coil conductor 112a and a transition 114 of the second coil conductor 112b. Alternatively, the transitions 114 of the second coil conductor 112b may be axially offset with respect to the transitions 114 of the first coil conductor 112a. This may reduce the effect of induced currents in portions of the first and second coil conductors 112a, 112b that occur before being cancelled at a respective transition 114, for example, by reducing overlapping induced currents. As discussed herein, the approach of including one or more transitions in a coil conductor may be used to reduce or even eliminate the adverse effects of currents that would otherwise be induced in leads exposed to magnetic and/or electric fields. It should be understood that the approach described herein may be combined with any one or more of the approaches described in the incorporated copending applications referenced above in the “Cross-Reference to Related Application” section of this present patent application. For example, the helical coil pitch may vary along at least a portion of the coil conductor, increasing either proximally or distally. The lead may include one or more shield layers. Further, multiple coil conductors and/or filars may be made of different electrically conductive materials, may have opposing directions of increasing/decreasing helical coil pitch, and/or may have alternating relative radial positions. For any of the embodiments described above with respect to FIGS. 2-6, no coil loop may short circuit to an adjacent coil loop. Thus, the wires are insulated via their own insulation jackets or via the lead body material in which they are imbedded such that no coil loop is capable of electrically shorting to an adjacent coil loop. Thus, the electricity is forced to go through the entire length of the wire forming a coiled conductor, as opposed to shorting from coil loop to coil loop of the coiled conductor. Insulation for the wires may be in the form of electrical insulation coatings or jackets, which may be of materials such as, for example, ETFE, PTFE, etc.

It should be understood that the different embodiments described herein are not mutually exclusive or exhaustive of the embodiments contemplated. In particular, any of the features discussed with respect to one of the embodiments may be employed in combination with any of the features discussed with respect to other embodiments. Thus, although the present invention has been described with reference to preferred embodiments, persons skilled in the art will recognize that changes may be made in form and detail without departing from the spirit and scope of the invention.

What is claimed is:

1. An implantable medical lead for coupling to an implantable pulse generator and configured for improved MRI safety, the lead comprising:
a tubular body including a proximal end and a distal end;
a first electrode operably coupled to the tubular body near the distal end; and
a first electrical coil conductor extending distally through the body from the proximal end and electrically connected to the first electrode, the first coil conductor including at least one transition in which the coil conductor changes from being helically coiled in a first direction for a first portion of the first electrical coil to being helically coiled in a second opposite direction for a second portion of the first electrical coil distal to the first portion of the first electrical coil.

2. The lead of claim 1, wherein the first coil conductor includes a plurality of transitions in which the helical coiling of the coil conductor changes direction.

3. The lead of claim 2, wherein the plurality of transitions are evenly spaced along a length of the conductor.

4. The lead of claim 2, wherein the plurality of transitions are spaced less than five windings apart.

5. The lead of claim 2, wherein the plurality of transitions are spaced less than 1.5 windings apart.

6. The lead of claim 2, further comprising a plurality of reinforcement rings, each reinforcement ring being disposed over a respective one of the transitions.

7. The lead of claim 1, further comprising a reinforcement ring disposed over the at least one transition.

8. The lead of claim 7, wherein the reinforcement ring comprises a plastic material.

9. The lead of claim 7, wherein the reinforcement ring comprises a metal.

10. The lead of claim 1, wherein a helical angle of the first coil conductor changes at least at the at least one transition.

11. The lead of claim 1, wherein the first coil conductor comprises an insulated wire at least between the proximal and distal ends.

12. The lead of claim 1, further comprising a second electrode operably coupled to the tubular body near the distal end and a second electrical coil conductor extending distally through the body from the proximal end and electrically connected to the second electrode, the second coil conductor including at least one transition in which the coil conductor changes from being helically coiled in a first direction to being helically coiled in a second opposite direction.

13. The lead of claim 12, wherein the at least one transition of the second coil conductor is substantially axially aligned with the at least one transition of the first coil conductor.

14. The lead of claim 13, wherein the first coil conductor and the second coil conductor are helically coiled in opposite directions.

15. The lead of claim 13, further comprising a reinforcement ring disposed over the at least one transition of the first coil conductor and over the at least one transition of the second coil conductor.

16. The lead of claim 12, wherein the at least one transition of the second coil conductor is axially offset from the at least one transition of the first coil conductor.

17. The lead of claim 16, further comprising a first reinforcement ring disposed over the at least one transition of the first coil conductor and a second reinforcement ring disposed over the at least one transition of the second coil conductor.

18. The lead of claim 12, wherein the at least one transition of the first coil conductor comprises a plurality of transitions, the at least one transition of the second coil conductor comprises a plurality of transitions, and the transitions of the second coil conductor are substantially aligned with respective transitions of the first coil conductor.

19. The lead of claim 13, wherein the first coil conductor and the second coil conductor are helically coiled in opposite directions.

20. The lead of claim 12, wherein the at least one transition of the first coil conductor comprises a plurality of transitions, the at least one transition of the second coil conductor comprises a plurality of transitions, and the transitions of the second coil conductor are offset from the transitions of the first coil conductor.

21. A method of forming an implantable medical lead configured for improved MRI safety, the method comprising:
helically coiling at least a portion of a first electrical coil conductor by:
winding the first coil conductor in a first direction about a longitudinal axis of the medical lead for a first portion of the medical lead; and
winding the first coil conductor in a second direction about the longitudinal axis of the medical lead, the second direction being opposite the first direction so as to form a transition in the first coil conductor in which the first coil conductor changes from being helically coiled in the first direction to being helically coiled in the second direction for a second portion of the medical lead distal from the first portion of the medical lead; and
forming an implantable medical lead including the helically coiled first coil conductor.

22. The method of claim 21, wherein helically coiling at least a portion of the first electrical coil conductor comprises forming a plurality of transitions in which the coil conductor changes direction of helical coiling.

23. The method of claim 21, further comprising changing a helical coil pitch of the first coil conductor at least at the at least one transition.

24. The method of claim 21, further comprising:
helically coiling at least a portion of a second electrical coil conductor by:
winding the second coil conductor in a first direction; and
winding the second first coil conductor in a second direction opposite the first direction so as to form a transition in the second coil conductor in which the second coil conductor changes from being helically coiled in the first direction to being helically coiled in the second direction; and
forming the implantable medical lead including the helically coiled second coil conductor.

25. The method of claim 24, wherein forming the implantable medical lead including the helically coiled second coil conductor comprises axially aligning the at least one transition of the second coil conductor substantially with the at least one transition of the first coil conductor.
